(19) United States
(12) Patent Application Publication
(10) Pub. No.: US 2023/0190778 A1
(43) Pub. Date: Jun. 22, 2023

(54) COMPOSITION COMPRISING GLUCOSE OLIGOSACCHARIDE AND PROCESS FOR MAKING THE SAME AND USE THEREOF

(71) Applicant: CARGILL, INCORPORATED, Wayzata, MN (US)

(72) Inventors: Jean E. DE OLIVEIRA, Vilvoorde (BE); Ronny Leontina Marcel VERCAUTEREN, Beveren (BE)

(73) Assignee: CARGILL, INCORPORATED, Wayzata, MN (US)

(21) Appl. No.: 17/999,613

(22) PCT Filed: May 28, 2021
(86) PCT No.: PCT/US2021/034717

§ 371 Date: Nov. 22, 2022

(30) Foreign Application Priority Data
May 29, 2020 (EP) 20177583.0

(51) Int. Cl. A61K 31/702; A23K 20/163; A23K 50/40; A61K 9/00; A61P 39/00

(52) U.S. Cl. CPC A61K 31/702; A23K 20/163; A23K 50/40; A61K 9/0056; A61P 39/00

(57) ABSTRACT

The present invention relates to a composition comprising glucose oligosaccharide and a process for making such. The present invention further relates to a composition comprising glucose oligosaccharide for use in the treatment of an animal. The present invention further relates to the use of a composition comprising glucose oligosaccharide to improve the growth performance of an animal. The present invention further relates to the use of a composition comprising glucose oligosaccharide to improve the immune system modulation of an animal.

### CROSS-REFERENCE TO RELATED APPLICATIONS

This application claims the benefit of European Application No. 20177583.0, filed May 29, 2020, and entitled “GLUCOSE-OLIGOSACCHARIDE COMPOSITIONS AND USES THEREOF”, which is incorporated by reference herein in its entirety.

### FIELD OF THE INVENTION

The present invention relates to a composition comprising glucose oligosaccharide and a process for making such a composition. The present invention further relates to animal feed comprising the composition of the present invention. The present invention further relates to the use of a composition according to the present invention for the prophylactic treatment of animals, for instance for improving disease resistance, and/or immune system modulation. The present invention further relates to the use of a composition according to the present invention for increasing growth performance in animals, in particular under challenging conditions.

### BACKGROUND OF THE INVENTION

There is an existing demand for nutritional supplements for improving overall health, in particular the immune system, of farm animals, including farmed fish and seafood, and of pets.

With the world population increasing, the demand for animal proteins for human consumption is ever growing. The animal production industry is facing the challenge to breed more animals and this is done by increasing the animal population but also by increasing animal growth performance and improving animal health in general. Animal health and animal growth performance are key factors for successful animal farming.

In particular, the modulation of the immune system is an essential factor when it comes to animal growth performance, particularly under challenging conditions, and feed additives are used to achieve this. Another main issue is bacterial contamination, particularly true in poultry and pigs. A stronger immune system can help to fight off such bacterial infections, rather than resorting to the use of antibiotics, which results in the development of antibiotic resistance of the bacteria over time.

Some oligosaccharide compositions are known to improve overall health, in particular gut health, for instance some prebiotic existing manno-oligosaccharide (or mannose oligosaccharide, or mannan-oligosaccharide or MOS) and fructo-oligosaccharide (FOS) and galacto-oligosaccharides (GOS). However, these have not been shown to have any beneficial impact directly on the modulation of the immune system.

In addition, they all present the disadvantage of being difficult to either isolate from nature e g manno- oligosaccharides from yeast cell walls, or require extensive chemical synthesis and purification e.g. FOS produced by inulin degradation enzymatically or chemically or FOS produced by transfructosylation action of a β-fructosidase of Aspergillus on sucrose. Thus, these products are too costly, generating many waste streams and a non-negligible environmental impact, when used commercially in feed compositions.

There is thus clearly still a need to provide oligosaccharides that can modulate the immune system, preferably using more than one mechanism of the immune system. There is thus also clearly still a need to provide more efficient methods for producing dietary fibres, such as oligosaccharides. Also, there is a need to provide oligosaccharides that are produced in a more economical environmentally friendly way. The present invention attempts to address those needs.

### SUMMARY OF THE INVENTION

In a first aspect, the present invention relates to a composition comprising glucose oligosaccharide (“glucose oligosaccharide composition of the present invention”) obtainable by a process comprising the aqueous polymerisation of glucose at a concentration of 50 to 95%, preferably 70 to 90% (dry substance) in the presence of a hydrochloric acid catalyst at a concentration of 0.01 to 0.25M, preferably 0.10 to 0.2M, more preferably 0.10 to 0.15M, at a temperature of from 50 to 120° C., preferably 50 to 99° C.

In a further aspect, the present invention relates to an animal feed or pet food product comprising the glucose oligosaccharide composition of the present invention and further animal feed or pet food ingredients.

In a further aspect, the present invention relates to a process for making a composition comprising glucose oligosaccharide characterized in that it comprises the steps of
a) Preparing an aqueous solution of glucose having a concentration of 50 to 95%, preferably 70 to 90% (dry substance),
b) Adding hydrochloric acid catalyst to the aqueous solution of glucose to reach a concentration of 0.01 to 0.25M, preferably 0.10 to 0.2M, more preferably 0.10 to 0.15M, hydrochloric acid,
c) Bringing the solution to a temperature of from 50 to 120° C., preferably 50 to 99° C. to polymerise the glucose to obtain a glucose oligosaccharide composition, and
d) Optionally, adjusting the pH of the glucose oligosaccharide composition to a pH of from 4 to 7.

In a further aspect, the present invention relates to the use of the glucose oligosaccharide composition of the present invention for improving the growth performance of an animal or pet, in particular under challenging conditions.

In a further aspect, the present invention relates to the use of the glucose oligosaccharide composition of the present invention for improving the immune system modulation of an animal or pet.

Finally, the present invention also relates to the use of the glucose oligosaccharide composition of the present invention for the prophylactic treatment of an animal or pet to prevent or reduce the severity of a disease, in particular an infectious disease.

### FIGURES

FIG. 1 shows the HPLC analysis of the glucose oligosaccharide according to the invention.

FIG. 2 shows the Nitric oxide (NO)-production assay results.

FIG. 3 shows the phagocytosis assay results.

FIG. 4 shows the in ovo trial results.

FIG. 5 shows set and measured temperatures in the Poultry Metabolic facility during the experimental period.

FIG. 6 shows an increase in the intestinal lactobacillus population of chickens fed with a diet containing the composition according to the invention.

### DETAILED DESCRIPTION

The use of “a” or “an” to describe the various elements or components herein is merely for convenience and to give a general sense of the invention. This description should be read to include one or at least one, and the singular also includes the plural unless it is obvious that it is meant otherwise.

All weight percentages disclosed herein are on a dry basis (db) unless indicated otherwise.

### The Glucose Oligosaccharide

In the present description, the terms glucose oligosaccharide and gluco-oligosaccharide are used interchangeably to describe the same oligosaccharide. Glucose oligosaccharide is defined as an oligosaccharide of glucose having a DP of 3 or more. DP refers to the degree of polymerisation, i.e. the number of monomers present in the oligosaccharide. In glucose oligosaccharide, the monomer is glucose. The glucose oligosaccharide composition of the present invention comprises glucose oligosaccharides having a DP of 3 or more and further it may comprise glucose monosaccharide and disaccharide. HPLC analysis (ISO 10504:1998-10) may be done to determine the amount and type of the various saccharide present in the glucose oligosaccharide composition, such as DP1, DP2, DP3 and higher.

The invention is defined in the included clauses and appended claims. At least one aspect of the invention is based on the finding that the composition comprising glucose oligosaccharide according to the present invention (“composition of the present invention” or “glucose oligosaccharide composition of the present invention”) has improved effects in an animal, compared to existing oligosaccharides compositions. The improved effects of the glucose oligosaccharides composition of the present invention are, amongst others, an improved effect on growth performance in animals (in particular under challenging conditions), related in particular to an improved modulation of the immune system in animals.

Further, the glucose oligosaccharide composition is characterized in that it is not isolated or derived directly from yeast or plants. However, the glucose used to prepare the glucose oligosaccharide composition according to the invention may be derived from starch obtained from plants, such as corn or wheat.

The glucose oligosaccharide composition of the present invention may have one or more of the following features combined:
i. The glucose oligosaccharide composition of the present invention may have a content of glucose oligosaccharides having a DP of at least 3 of from 30 weight % (wt %) or higher on a dry basis (db), preferably from 40 wt % db or higher. More preferably, the content of glucose oligosaccharides having a DP of at least 3 may be from 40 wt % to 55 wt % db, most preferably from 40 wt % to 50 wt % db of the composition. The glucose oligosaccharide composition of the present invention may have a content of glucose oligosaccharides having a DP of at least 3 of about 45 wt % db.
ii. Further, the glucose oligosaccharide composition may contain glucose in an amount of from 10 to 60 wt % db, preferably from 15 to 55 wt % db, more preferably 20 to 50 wt % db, most preferably 30 to 48 wt % db. The content of glucose may be from about 30 to 47 wt % db. The content of glucose may be from about 40 to 47 wt % db. The content of glucose may be from about 42 to 47 wt % db. The content of glucose may be from about 44 to 47 wt % db. The content of glucose may be about 45 wt % db.
iii. The glucose oligosaccharide composition of the present invention also preferably comprises glucose disaccharide (DP2). Thus, the glucose oligosaccharide composition of the present invention may have a content of glucose dissaccharides of from 5 to 15 wt % db, preferably of from 6 to 12 wt % db, more preferably of from 7 to 10 wt % db, most preferably of from about 8 to 9 wt % db. The content of glucose disaccharides may be about 8.5 wt % db.
iv. Further, the glucose oligosaccharide composition may contain glucose trisaccharides

(DP3) in an amount of from 5 to 30 wt % db, preferably from 8 to 25 wt % db, more preferably 10 to 20 wt % db, most preferably 12 to 18 wt % db. The content of DP3 may be from about 12 to 15 wt % db. The content of glucose oligosaccharides having a DP3 may be from about 13 to 15 wt % db. The content of glucose oligosaccharides having a DP3 may be about 14 wt % db.
v. Further, the glucose oligosaccharide composition may contain glucose tetrasaccharides (DP4) in an amount of from 5 to 20 wt % db, preferably from 6 to 18 wt % db, more preferably 7 to 15 wt % db, most preferably 8 to 12 wt % db. The content of DP4 may be from about 9 to 10 wt % db. The content of glucose oligosaccharides having a DP4 may be about 9.5 wt % db.
vi. Further, the glucose oligosaccharide composition may contain glucose pentasaccharides (DP5) in an amount of from 3 to 20 wt % db, preferably from 4 to 18 wt % db, more preferably 5 to 15 wt % db, most preferably 6 to 12 wt % db. The content of glucose oligosaccharides having a DP5 may be from about 6 to 10 wt % db. The content of glucose oligosaccharides having a DP5 may be from about 6 to 8 wt % db. The content of glucose oligosaccharides having a DP5 may be from about 6 to 7 wt % db. The content of glucose oligosaccharides having a DP5 may be about 6.5 wt % db.
vii. Further, the glucose oligosaccharide composition may contain glucose oligosaccharides having a DP6 in an amount of from 2 to 15 wt % db, preferably from 3 to 12 wt % db, more preferably 3 to 10 wt % db, most preferably 4 to 8 wt % db. The content of glucose oligosaccharides having a DP6 may be from about 4 to 6 wt % db. The content of glucose oligosaccharides having a DP6 may be from about 4 to 5 wt % db. The content of glucose oligosaccharides having a DP6 may be about 4.5 wt % db.
viii. Further, the glucose oligosaccharide composition may contain glucose oligosaccharides having a DP7 in an amount of from 2 to 15 wt % db, preferably from 3 to 12 wt % db, more preferably 3 to 10 wt % db, most preferably 4 to 8 wt % db. The content of DP7 may be from about 4 to 6 wt % db. The content of glucose oligosaccharides having a DP7 may be from about 4 to 5 wt % db. The content of glucose oligosaccharides having a DP7 may be about 4.5 wt % db.
ix. Further, the glucose oligosaccharide composition may contain glucose oligosaccharides having a DP8 in an amount of from 1 to 10 wt % db, preferably from 1.5 to 8 wt % db, more preferably 2 to 6 wt % db, most preferably 2 to 4 wt % db. The content of glucose oligosaccharides having a DP8 may be about 2 wt % db.
x. Further, the glucose oligosaccharide composition may contain glucose oligosaccharides having a DP9 in an amount of from 0.5 to 5.0 wt % db, preferably from 0.8 to 4.0 wt % db, more preferably 1.0 to 3.0 wt % db, most preferably 1.0 to 2.0 wt % db. The content of glucose oligosaccharides having a DP9 may be about 1.5 wt % db.
xi. Further, the glucose oligosaccharide composition may contain glucose oligosaccharides having a DP greater than 9 in an amount of from 1 to 10 wt % db, preferably from 2 to 8 wt % db, more preferably 3 to 5 wt % db, most preferably 3 to 4 wt % db. The content of glucose oligosaccharides having a DP9 may be about 3.5 wt % db.
xii. The dry substance of the glucose oligosaccharide composition may be at least 70 wt %, preferably at least 75 wt %, more preferably at least 80 wt %, even more preferably at least 85 wt %, most preferably at least 90 wt %. The dry substance can be for example from 70 wt % to 90 wt %, or from 75 wt % to 85 wt %, or from 80 to 85 wt %.
xiii. Further, the glucose oligosaccharide composition may be characterised in that the glucose oligosaccharide comprises mainly alpha- & beta-1,6 glycosidic linkages. Preferably at least 45%, more preferably at least 50%, most preferably about 55% of the total number of glycosidic linkages are alpha- & beta-1,6 linkages (excluding the linkage in the non-reducing terminal residue). Preferably at most 67%, more preferably at most 65% or at most 60%, most preferably at most 55% of the total number of glycosidic linkages are alpha- & beta-1,6 linkages (excluding the linkage in the non-reducing terminal residue).
xiv. Further, the glucose oligosaccharide composition may be characterised in that the glucose oligosaccharide comprises alpha- & beta-1,4 glycosidic linkages. Preferably at least 5%, more preferably at least 7%, most preferably about 8% of the total number of glycosidic linkages are alpha- & beta-1,4 linkages (excluding the linkage in the non-reducing terminal residue). Preferably at most 15%, more preferably at most 12%, most preferably at most 11% or at most 10% of the total number of glycosidic linkages are alpha- & beta-1,4 linkages (excluding the linkage in the non-reducing terminal residue).
xv. Further, the glucose oligosaccharide composition may be characterised in that the glucose oligosaccharide comprises alpha- & beta-1,3 glycosidic linkages (excluding the linkage in the non-reducing terminal residue). Preferably at least 5%, more preferably at least 7%, most preferably about 8% of the total number of glycosidic linkages are alpha- & beta-1,3 linkages. Preferably at most 15%, more preferably at most 12%, most preferably at most 11% or at most 10% of the total number of glycosidic linkages are alpha- & beta-1,3 linkages (excluding the linkage in the non-reducing terminal residue).
xvi. Further, the glucose oligosaccharide composition may be characterised in that the glucose oligosaccharide comprises alpha- & beta-1,2 glycosidic linkages (excluding the linkage in the non-reducing terminal residue). Preferably at least 8%, more preferably at least 10%, most preferably at least 12% of the total number of glycosidic linkages are alpha- & beta-1,2 linkages. Preferably at most 18%, more preferably at most 15%, most preferably at most 14% of the total number of glycosidic linkages are alpha- & beta-1,2 linkages (excluding the linkage in the non-reducing terminal residue).
xvii. Further, the glucose oligosaccharide composition may be characterised in that the glucose oligosaccharide comprises alpha- & beta-1,3,6 or 1,2,6 glycosidic linkages. Preferably at least 4%, more preferably at least 5%, most preferably about 6% of the of the total number of glycosidic linkages are alpha- & beta-1,3,6 or 1,2,6 linkages (excluding the linkage in the non-reducing terminal residue). Preferably at most 12%, more preferably at most 10%, most preferably at most 8% of the total number of glycosidic linkages are alpha- & beta-1,3,6 or 1,2,6 linkages (excluding the linkage in the non-reducing terminal residue).

In all cases above, the term “about” means +/−0.5. The glucose oligosaccharide composition according to the invention hereby explicitly includes any combination of one or more of the features of (i) to (xvii) mentioned above.

The amount of linkages was determined according to the following PMAA method (based on Leeuwen et al. Carbohydrate Research 343 (2008) pp. 1237-1250, and according to the permethylation method described by Ciucanu et al. Carbohydrate Research (1984), 131, pp. 209-217):
The syrup samples were dissolved in 0.5mL water and then freeze-dried.
The first step in the linkage analysis was de permethylation of free hydroxyl groups.
The permethylated compound was hydrolysed with a 6M TFA solution to create partially methylated monosaccharides, followed by a reduction and an acetylation.
The (volatile) partially methylated monosaccharides were then analysed by GC-MS which gave rise to characteristic mass spectra and retention times. All measurements were performed in triplicate.
As the samples contained 30-40% free glucose, the peak for glucose was excluded for the calculation of the percentage of each glycosidic linkage type in the samples.

The glucose oligosaccharide composition according to the invention is normally used in a liquid form as a syrup. However, the dry substance of the glucose oligosaccharide composition can be adapted to the needs of its application. The glucose oligosaccharide composition can thus also be dried and stored in powder form depending on the intended use. In powder form, the glucose oligosaccharide composition of the present invention is a stable, yellowish to white, free flowing powder.

### Process for Preparing the Glucose Oligosaccharide Composition of the Invention

The invention further relates to a process for making a glucose oligosaccharide composition, said process comprises the steps of:
a) Preparing an aqueous solution of glucose having a concentration of 50 to 95 wt %, preferably 70 to 90 wt %,
b) Adding hydrochloric acid catalyst to the aqueous solution of glucose to reach a concentration of 0.01 to 0.25M hydrochloric acid, preferably 0.10 to 0.2M, more preferably 0.10 to 0.15M,
c) Bringing the solution to a temperature of from 50 to 120° C., preferably 50 to 99° C., to polymerise the glucose to obtain a glucose oligosaccharide composition, and
d) Optionally, adjusting the pH of the glucose oligosaccharide composition to a pH of from 4 to 7.

All combinations of one or more of the preferred features of the process according to the invention below are hereby explicitly included.

It is advantageous that the aqueous glucose solution comprises from 50 to 95 wt % or 50 to 90 wt % of glucose, preferably from 60 to 89 wt %, more preferably from 70 to 88 wt %, even more preferably from 80 to 87 wt %, most preferably about 85 wt % of glucose (dry substance basis). The preferred method is that the aqueous solution essentially consists of only water and glucose. This can be prepared by dissolving a high purity (e.g. min. 95 or 99 wt %) crystalline glucose (also known as dextrose) in water. A preferred crystalline glucose is crystalline a-D-glucose (dextrose) monohydrate, for instance C*Dex™ 02001 from Cargill.

It is advantageous that the concentration of hydrochloric acid (which acts as the catalyst) in the aqueous solution from step b is from 0.01 to 0.25 M, preferably 0.10 to 0.2 M, more preferably 0.10 to 0.15 M, most preferably about 0.1 M.

It is advantageous that the temperature of step c) is from 50 to 120° C., preferably 50 to 99° C., preferably from 60 to 98° C., more preferably from 70 to 95° C., even more preferably from 80 to 92° C., yet more preferably from 85 to 92° C., most preferably around 90° C.

The reaction time for the polymerisation of glucose in step c) is from 2.5 to 40 hours, preferably 5 to 30 hours, preferably from 10 to 27 hours, more preferably from 12 to 25 hours, even more preferably from 15 to 23 hours, most preferably from 16 to 20 hours. The reaction time for the polymerisation of glucose in step c) can be about 18 hours. The reaction time will depend on the temperature and the concentration of the hydrochloric acid. The higher the temperature and the higher the concentration of the hydrochloric acid catalyst, the shorter the required reaction time.

Preferably the glucose oligosaccharide composition is neutralised. Neutralising the glucose oligosaccharide composition may be done until the composition reaches a pH of from 4 to 7. This is advantageous for an increased stability of the product, e.g. less hydrolysis over time and thus less to no change in composition of the product. Also, an advantage is that the product is then suitable for use with other ingredients that are sensitive to acids or acidic conditions. Neutralisation may be done with any suitable base. Preferably, in particular when the glucose oligosaccharide composition is to be used in feed and pet food, the base is caustic and/or potassium hydroxide.

It should be noted that it is possible to either neutralise the glucose oligosaccharide composition or to decolorize the glucose oligosaccharide composition or to do both.

Preferably the glucose oligosaccharide composition is refined. Refining the produced glucose oligosaccharide composition may be done by passing it over series of anionic and cationic resins, and/or a polisher such as active carbon and/or chromatography. In particular refining by chromatography may be done to remove part or all glucose from the composition, if required.

The present invention further relates to a glucose oligosaccharide composition obtainable by the process of the present invention. Indeed such glucose oligosaccharide composition has, amongst others, the improved effects as discussed herein.

### Use in Feed and Pet Food and Effects in Animals

The glucose oligosaccharide composition may be used as a feed additive or a feed ingredient in an animal feed and pet food product.

The glucose oligosaccharide compositions of the present invention have an improved effect on the growth performance of animals, in particular under challenging conditions. The glucose oligosaccharide compositions of the present invention can thus be used to improve the growth performance of animals (i e in comparison to animals fed without the composition according to the invention), in particular under challenging conditions. The invention also covers the glucose oligosaccharide compositions of the present invention for use in improving the growth performance of animals, in particular under challenging conditions. By “challenging conditions” it is herein meant conditions of diseases, in particular infectious diseases, and also conditions that cause stress, such as environmental stress (heat, humidity etc.), transport stress, behavioural stress, stress from being vaccinated or from being manipulated. Infectious diseases are caused by pathogens e.g. viruses, bacteria, fungi, parasites and the like.

The present invention further relates to the use of the glucose oligosaccharide composition as described herein as a prophylactic medicament for animals The invention also covers the glucose oligosaccharide composition as described herein for use as a medicament for prophylactically treating animals. By “prophylactic”, it is meant herein to guard against or prevent disease and also to reduce the severity of a disease. In particular, the invention relates to the use of the glucose oligosaccharide composition as a prophylactic medicament for animals against diseases, in particular infectious diseases. The invention also covers the glucose oligosaccharide composition as described herein for use as a medicament for prophylactically treating animals against diseases, in particular infectious diseases. Infectious diseases are caused by pathogens e.g. viruses, bacteria, fungi, parasites and the like.

It has been found that the glucose oligosaccharide composition according to the present invention is able to modulate the immune system, i.e. able to modulate the response of immune cells or macrophages. Therefore, the present invention further relates to a glucose oligosaccharide composition according to the present invention for use in modulating the response of immune cells or macrophages. The present invention further relates to the use of a glucose oligosaccharide composition according to the present invention to modulate the response of immune cells or macrophages.

The glucose oligosaccharide composition according to the present invention modulates the immune system by two different mechanisms, allowing a dual action and increased performance of the glucose oligosaccharide composition compared to other known fibre-like oligosaccharides e.g. mannose oligosaccharides.

The glucose oligosaccharide composition according to the present invention is able to modulate the NO production of macrophages (as shown in nitric oxide (NO) in vitro assays). A certain increase in NO production helps in cytotoxic and defence mechanisms against viruses, tumour cells, bacteria, fungi, protozoa and helminths. This results in protection of the host against infection and initiates activation of the innate immune system. However, too high a production of NO has suppressive effects on lymphocyte proliferation and may cause damage to other normal host cells. The glucose oligosaccharide composition is able to stimulate the right amount of NO production by the macrophages i.e. to modulate this immune response.

The glucose oligosaccharide composition according to the present invention is able to increase phagocytosis in macrophage cells (as shown in phagocytosis in vitro assays). With the help of the glucose oligosaccharide composition macrophages can increase internalization of external bacteria (phagocytosis). Without being bound by theory, these internalized bacteria are presented to other immune cells, such as B and T cells. This results in activation of the innate immune system and can subsequently activate the adaptive immune system by presentation of bacterial peptides.

The present invention further relates to the use of the glucose oligosaccharide composition for improving the gut health of an animal, in particular increasing the population of lactobacillus bacteria in the digestive system of an animal

The present invention further relates to the use of the glucose oligosaccharide composition as described herein for improving weight gain and/or increasing feed intake and/or increasing body weight of animals This includes both the therapeutic and non-therapeutic use thereof in improving weight gain and/or increasing feed intake and/or increasing body weight in animals The invention includes the glucose oligosaccharide composition as described herein for therapeutic use in improving weight gain and/or increasing feed intake and/or increasing body weight of animals.

Preferably the animal is poultry, pig, ruminants, horses, aquatic animals (such as fish, e.g. salmon or trout, and shrimps), or pets. More preferably the animal is poultry or pig. Poultry includes broiler chickens, turkeys, ducks, or geese. It has been shown, exemplified in the example section below, that surprisingly the intake of glucose oligosaccharide composition according to the present invention is beneficial to the immune systems of the animals, thereby guarding against disease and in turn increasing the growth performance of the animals, in particular under challenging conditions. Furthermore, in in-ovo administration of the compound in several doses it has been shown that the glucose oligosaccharide according to the invention is safe for use in the feed of animals. The composition according to the invention did not cause any mortalities.

All of the above uses also apply to animal feed or pet food products comprising the glucose oligosaccharide composition according to the present invention and other feed or pet food ingredients, respectively.

### The Feed Composition

The present invention further relates to an animal feed, or pet food product comprising the glucose oligosaccharide composition of the present invention and further animal feed or pet food ingredients.

The animal feed product may be a feed product designed for feeding pig, preferably piglet, or chicken, such as a feed for broilers or a feed for layers, or fish feed, such as salmon or trout. The animal feed may also be horse feed. Said pig feed, chicken feed, fish feed or horse feed comprise next to the glucose oligosaccharide composition of the present invention, feed ingredients typically or specifically used for such animal feed. The person skilled in the art of preparing animal feed is aware of typical and specific compositions of animal feed. The glucose oligosaccharide composition is also very suitable to be used as a feed supplement. As it is soluble, it can easily be added to the drinking water for example. Further feed ingredients of the feed product may be other carbohydrates and fibres such as glucans, arabinoxylan oligosaccharides, proteins, fats, vitamins, minerals and the like. Depending on the animal species, the composition of the feed will vary; the skilled person understands how to formulate suitable feed compositions.

Preferably, the glucose oligosaccharide composition is present in the animal feed or pet food product in such an amount as to provide from 0.01 to 20 g, preferably from 0.01 to 10 g of the glucose oligosaccharide composition per kg of body weight of the animal or pet per day, in one or more servings. Preferably, the glucose oligosaccharide composition is present in the animal feed or pet food product in such an amount as to provide from 0.01 to 0.02 g of the glucose oligosaccharide composition per kg of body weight of the animal or pet per day, in one or more servings. In particular, the animal feed or pet food may comprise from 0.02 to 0.6 wt % of the present glucose oligosaccharide composition, based on the weight of the animal feed or pet food product.

In one aspect, the animal feed or pet food, preferably pig feed comprises from 0.1 to 0.5 wt %, preferably from 0.1 to 0.4 wt %, even more preferably from 0.1 to 0.3 wt %, yet even more preferably from 0.1 to 0.2 wt % of the glucose oligosaccharide composition of the present invention, based on the weight of the animal feed or pet food product.

In another aspect, the animal feed or pet food, preferably poultry feed comprises from 0.01 to 1.0 wt %, preferably from 0.01 to 0.5 wt %, even more preferably from 0.01 to 0.3 wt %, yet even more preferably from 0.01 to 0.2 wt %, most preferably from 0.02 to 0.2 wt % of the glucose oligosaccharide composition of the present invention, based on the weight of the animal feed or pet food product.

### CLAUSES OF THE INVENTION

The invention covers the following:

Clause 1: A glucose oligosaccharide composition obtainable by a process comprising the aqueous polymerisation of glucose at a concentration of 50 to 95 wt %, preferably 70 to 90 wt %, in the presence of hydrochloric acid at a concentration of 0.01 to 0.25M, preferably 0.10 to 0.20 M, more preferably 0.10 to 0.15 M, at a temperature of from 50 to 120° C., preferably 50 to 99° C.

Clause 2: The composition according to clause 1 characterized in that it comprises of from 30 weight % (wt %) on a dry basis (db) or higher, preferably from 40 wt % db or higher, more preferably about 45 wt % db of glucose oligosaccharides having a degree of polymerisation (DP) of at least 3.

Clause 3: The composition according to any one of the previous clauses characterized in that it comprises glucose in an amount of from 10 to 60 wt % db, preferably from 15 to 55 wt % db, more preferably 20 to 50 wt % db, most preferably 30 to 48 wt % db.

Clause 4: The composition according to any one of the previous clauses characterized in that it comprises glucose disaccharides in an amount of from 5 to 15 wt % db, preferably of from 6 to 12 wt % db, more preferably of from 7 to 10 wt % db, most preferably of from about 8 to 9 wt % db.

Clause 5: The composition according to any one of the previous clauses characterized in that it comprises glucose oligosaccharides having a DP of 3 in an amount of from 5 to 30 wt % db, preferably from 8 to 25 wt % db, more preferably 10 to 20 wt % db, most preferably 12 to 18 wt % db.

Clause 6: The composition according to any one of the previous clauses characterized in that it comprises glucose oligosaccharides having a DP of 4 in an amount of from 5 to 20 wt % db, preferably from 6 to 18 wt % db, more preferably 7 to 15 wt % db, most preferably 8 to 12 wt % db.

Clause 7: The composition according to any one of the previous clauses characterized in that it comprises glucose oligosaccharides having a DP of 5 in an amount of from 3 to 20 wt % db, preferably from 4 to 18 wt % db, more preferably 5 to 15 wt % db, most preferably 6 to 12 wt % db.

Clause 8: The composition according to any one of the previous clauses characterized in that it comprises glucose oligosaccharides having a DP of 6 in an amount of from 2 to 15 wt % db, preferably from 3 to 12 wt % db, more preferably 3 to 10 wt % db, most preferably 4 to 8 wt % db.

Clause 9: The composition according to any one of the previous clauses characterized in that it comprises glucose oligosaccharides having a DP of 7 in an amount of from 2 to 15 wt % db, preferably from 3 to 12 wt % db, more preferably 3 to 10 wt % db, most preferably 4 to 8 wt % db.

Clause 10: The composition according to any one of the previous clauses characterized in that it comprises glucose oligosaccharides having a DP of 8 in an amount of from 1 to 10 wt % db, preferably from 1.5 to 8 wt % db, more preferably 2 to 6 wt % db, most preferably 2 to 4 wt % db.

Clause 11: The composition according to any one of the previous clauses characterized in that it comprises glucose oligosaccharides having a DP of 9 in an amount of from 0.5 to 5.0 wt % db, preferably from 0.8 to 4.0 wt % db, more preferably 1.0 to 3.0 wt % db, most preferably 1.0 to 2.0 wt % db.

Clause 12: The composition according to any one of the previous clauses characterized in that it comprises glucose oligosaccharides having a DP greater than 9 in an amount of from 1 to 10 wt % db, preferably from 2 to 8 wt % db, more preferably 3 to 5 wt % db, most preferably 3 to 4 wt % db.

Clause 13: The composition according to any one of the previous clauses characterized in that it comprises a dry substance of at least 70 wt %, preferably at least 75 wt %, more preferably at least 80 wt %.

Clause 14: The composition according to any one of the previous clauses characterised in that at least 45%, preferably at least 50%, and at most 67%, preferably at most 65% or at most 60%, of the total number of glycosidic linkages of the glucose oligosaccharides are alpha- & beta-1,6 linkages, wherein the “total number of glycosidic linkages” excludes linkages in the non-reducing terminal residues

Clause 15: The composition according to any one of the previous clauses characterised in that at least 5%, more preferably at least 7%, and at most 15%, preferably at most 12%, more preferably at most 11% or at most 10% of the total number of glycosidic linkages of the glucose oligosaccharides are alpha- & beta-1,4 linkages (wherein the “total number of glycosidic linkages” excludes linkages in the non-reducing terminal residues).

Clause 16: The composition according to any one of the previous clauses characterised in that at least 5%, more preferably at least 7%, and at most 15%, preferably at most 12%, more preferably at most 11% or at most 10% of the total number of glycosidic linkages of the glucose oligosaccharides are alpha- & beta-1,3 linkages (wherein the “total number of glycosidic linkages” excludes linkages in the non-reducing terminal residues).

Clause 17: The composition according to any one of the previous clauses characterised in that at least 8%, preferably at least 10%, more preferably at least 12%, and at most 18%, preferably at most 15%, more preferably at most 14% of the total number of glycosidic linkages of the glucose oligosaccharides are alpha- & beta-1,2 linkages (wherein the “total number of glycosidic linkages” excludes linkages in the non-reducing terminal residues).

Clause 18: The composition according to any one of the previous clauses characterised in that at least 4%, preferably at least 5%, and at most 12%, preferably at most 10%, of the total number of glycosidic linkages of the glucose oligosaccharides are alpha- & beta-1,3,6 or 1,2,6 linkages (wherein the “total number of glycosidic linkages” excludes linkages in the non-reducing terminal residues).

Clause 19: The composition according to any one of the previous clauses characterized in that the temperature of the polymerisation step in the process is from 60 to 98° C., preferably from 70 to 95° C., more preferably from 80 to 92° C., yet more preferably from 85 to 92° C., most preferably around 90° C.

Clause 20: The composition according to any one of the previous clauses characterized in that the reaction time of the polymerisation step in the process is from 2.5 to 40 hours, preferably 5 to 30 hours, preferably from 10 to 27 hours, more preferably from 12 to 25 hours, even more preferably from 15 to 23 hours, most preferably from 16 to 20 hours or about 18 hours.

Clause 21: An animal feed or a pet food product comprising the composition of any one of clauses 1 to 20 and further animal feed or pet food ingredients. The feed product can be feed for poultry e.g. chicken, turkey, ducks, geese.

Clause 22: The animal feed or pet food product of clause 21, wherein the composition of any one of clauses 1 to 20 is present in an amount sufficient to provide from 0.01 to 0.02 g of the composition per kg of body weight of the animal or pet (per day), in one or more servings.

Clause 23: The animal feed or pet food product of clauses 21 or 22 wherein the composition of any one of clauses 1 to 20 is present in an amount of from 0.01 to 0.5 wt %, preferably from 0.01 to 0.3 wt %, more preferably from 0.02 to 0.2 wt %, based on the weight of the animal feed or pet food product.

Clause 24: Use of the glucose oligosaccharide composition according to any one of clauses 1 to 20 as a feed additive or feed ingredient in an animal feed or pet food product. The feed product can be feed for poultry e.g. chicken, turkey, ducks, geese.

Clause 25: Use of the composition according to any one of clauses 1 to 20 or the feed/pet food product according to any one of clauses 21 to 23 for improving the growth performance of animals, in particular under challenging conditions. The animal can be poultry e.g. chicken, turkey, ducks, geese. By “challenging conditions” it is herein meant conditions of diseases, in particular infectious diseases, and also conditions that cause stress, such as environmental stress (heat, humidity etc.), transport stress, behavioural stress, stress from being vaccinated or from being manipulated.

Clause 26: Use of the composition according to any one of clauses 1 to 20 or the feed/pet food product according to any one of clauses 21 to 23 for improving the immune system modulation of animals. The animal can be poultry e.g. chicken, turkey, ducks, geese.

Clause 27: The composition according to any one of clauses 1 to 20 or the feed/pet food product according to any one of clauses 21 to 23 for use in the prophylactic treatment of animals. The animal can be poultry e.g. chicken, turkey, ducks, geese.

Clause 28: The composition or feed/pet food product according to clause 27 wherein the prophylactic treatment prevents, or reduces the severity of, infectious diseases, preferably diseases caused by viruses, bacteria, fungi or parasites. The diseases can be diseases affecting poultry.

Clause 29: A process for making a composition comprising glucose oligosaccharides (preferably according to any one of clauses 1 to 20) characterized in that it comprises the steps of:
a) Preparing an aqueous solution of glucose having a concentration of 50 to 95%, preferably 70 to 90% (dry substance),
b) Adding hydrochloric acid catalyst to the aqueous solution of glucose to reach a concentration of 0.01 to 0.25M hydrochloric acid, preferably 0.10 to 0.20 M, more preferably 0.10 to 0.15 M.
c) Bringing the solution to a temperature of from 50 to 120° C., preferably 50 to 99° C. to polymerise the glucose, and
d) Optionally, adjusting the pH of the glucose oligosaccharide composition to a pH of from 4 to 7.

Clause 30: The process according to clause 29 characterized in that the concentration of hydrochloric acid of the solution from step b) is from 0.02 to 1.5 M, preferably from 0.05 to 1.0 M, more preferably from 0.08 to 0.8 M, most preferably about 0.1 M.

Clause 31: The process according to any one of clauses 29 or 30 characterized in that the temperature of the polymerisation in step c) is from 60 to 98° C., preferably from 70 to 95° C., more preferably from 80 to 92° C., yet more preferably from 85 to 92° C., most preferably around 90° C.

Clause 32: The process according to any one of clauses 29 to 31 wherein the reaction time of the glucose polymerisation in step c) is from 2.5 to 40 hours, preferably 5 to 30 hours, preferably 10 to 27 hours, more preferably from 12 to 25 hours, even more preferably from 15 to 23 hours, most preferably from 16 to 20 hours or about 18 hours.

Clause 33: The process according to any one of clauses 29 to 32 wherein the aqueous solution of glucose consists essentially of glucose and water.

Clause 33: The process according to any one of clauses 29 to 32 wherein the aqueous solution of glucose is prepared prior to step (a) by dissolving crystalline glucose, having a purity of at least 95 wt %, preferably at least 99 wt %, in water.

The present invention will be illustrated by the following non-limiting examples.

### EXAMPLES

### Experiment 1: Production of Glucose Oligosaccharide Composition According to the Invention

### Example 1

1. Preparation of the syrup at 85% dry substance (ds)

The dry substance of the glucose monohydrate powder (Cargill C*Dex 02001) was determined using an IR balance at 105° C., 30 min: 91.4% ds.

The reactor was heated to 90° C.

The amount of required demineralized water to reach 85% ds was weighed and transferred to the reactor. The amount of required glucose monohydrate to reach 85% ds was weighed and added slowly spoon by spoon to the reactor whilst stirring to get a homogeneous mixture.

The dry substance of the resulting glucose syrup was measured by refractive index: 85% ds.

2. Condensation reaction (Dx syrup 85% ds, in 0.1M HCl at 90° C. treatment for 18 h)

Concentrated HCl 37% to obtain a HCl concentration in the reaction mixture of 0.1 M (i.e. 10 ml HCl 37%/1 syrup) was added to the 85% ds dextrose syrup. The temperature of the reaction was controlled at 90C and stirred for 18 hours. The reaction was mixture was then cooled to 60C.

3. Active carbon treatment (reaction at 30% ds, ratio carbon:syrup 1:7, 60° C., 2 h)

The active carbon required (the quantity needed is a carbon:syrup (at 30% ds) ratio of 1:7) was weighed. The active carbon was rinsed with demineralized water several times to remove the fine particles. The active carboned was decanted. The glucose oligosaccharide containing syrup (i.e. the composition according to the invention) was diluted in the reactor to 30% ds with demineralized water. The ds was measured and checked by refractive index. The decanted active carbon was added to the syrup in the reactor at 60° C. The mixture was gently stirred for 2 hours.

4. Filtration (Dicalite 478 filter aid)

A filter aid cake on a Buchner funnel was prepared and rinsed with demineralized water (for 2 L syrup having a 30% ds a Buchner funnel of 19 cm and about 60 g of filter aid was used). The rinsing liquid was discarded. The syrup was carefully poured onto the filter aid and the filtrate was collected in a clean filtration flask. The dry substance of the filtrate was measured by refractive index.

5. Syrup refining (cation, anion, polisher)

The refining columns were regenerated at 45° C. The following columns were connected in series: cation->anion->polisher. The dry substance and the conductivity of the syrup were measured.

The columns were heated to 60° C. and the syrup was pumped at a flow rate of 3-4 BV/h (for a cation column of 130 ml, a flowrate of 400 ml/h was applied). The refined syrup was collected when the ds reached greater than 0.5%. The total collection was mixed. The dry substance and the conductivity of the refined syrup were measured.

6. Additional filtration

Since the syrup was strongly coloured, the syrup was passed over GF/C glass fiber membrane 4× and 0.45 μm and 0.22 μm membranes. The colour was not further decreased, but the black spots resulting from the fines from the active carbon were removed.

7. Concentration of the refined syrup (Rotavapor)

The refined syrup was concentrated stepwise in a Rotavapor. The bath of the rotavapor was set at 60° C., the cooling bath at 10° C. The pressure was slowly decreased to avoid the syrup to boil over to the condensate flask. Finally, a vacuum of 30 mbar was applied. The syrup was concentrated to 85%ds (measured with refractive index). The sample was labelled EXAMPLE 1.

8. HPLC-Area % for oligosaccharides profile (Ag+column)

A sample of the syrup EXAMPLE 1. was diluted with demineralized water to about 10-15 % ds and filtered over 0.45 μm Sartorius disposable filter. The filtrate was collected in a HPLC vial and analyzed for oligosaccharides (2× Bio-Rad HPX-42A columns in series with de-ashing—using HPLC water as eluent at 0.6 mL/min 2 μl injection—using refractive index detector.). Rel. area % are shown in the following table:

| DP8+ | DP7 | DP6 | DP5 | DP4 | DP3 | DP2 | Unknown 1 | Dextrose | Fructose | Unknown 2 |
|---|---|---|---|---|---|---|---|---|---|---|
| 5.39 | 2.64 | 4.41 | 6.43 | 9.36 | 14.37 | 9.66 | 12.76 | 31.36 | 1.75 | 0.22 |

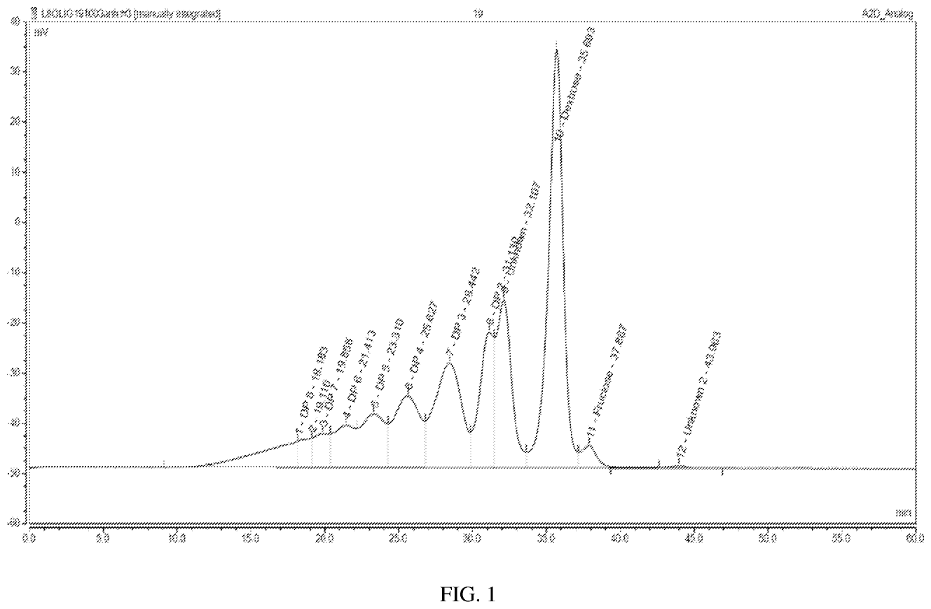

FIG. 1 shows the HPLC analysis.

9. GPC-low MW

A sample of the syrup EXAMPLE 1. was diluted with demineralized water to 10% ds and filtered over 0.45 μm Sartorius disposable filter. The filtrate was collected in a HPLC vial and analyzed with GPC for the low MW profile of the oligosaccharides. Two injections were carried out and the average was calculated. The GPC results are provided in the table below.

| | Injection 1 | Injection 2 | Average |
|---|---|---|---|
| Mn | 332 | 333 | 333 |
| Mw | 552 | 571 | 562 |
| Polydispersity (Mw/Mn) | 1.7 | 1.7 | 1.7 |

| | | Slicing | Slicing | Slicing |
|---|---|---|---|---|
| DP range | MW range | (Area %) | (Area %) | (Area %) |
| 1->5 | <909 | 82.6 | 82.6 | 82.6 |
| 6->9 | 909->1557 | 13.2 | 13.2 | 13.2 |
| 10->19 | 1557->3177 | 4.2 | 4.2 | 4.2 |
| 20->45 | 3177->7389 | 0.1 | 0.1 | 0.1 |

10. Linkages

The amount of different linkages of the syrup Example 1 was compared to those of commercially available soluble fibers Promitor® (resistant dextrin) and Litesse® (polydextrose).

The amount of linkages was determined according to the following PMAA method (based on Leeuwen et al. Carbohydrate Research 343 (2008) pp. 1237-1250, and according to the permethylation method described by Ciucanu et al. Carbohydrate Research (1984), 131, pp. 209-217):
1. The syrup from samples were dissolved in 0.5 mL water and then freeze-dried.
2. The first step in the linkage analysis was de permethylation of free hydroxyl groups.
3. The permethylated compound was hydrolysed with a 6M TFA solution to create partially methylated monosaccharides, followed by a reduction and an acetylation
4. The (volatile) partially methylated monosaccharides were then analysed by GC-MS which gave rise to characteristic mass spectra and retention times. All measurements were performed in triplicate.
5. As the samples contained 30-40% free glucose, the peak for glucose was excluded for the calculation of the percentage of each linkage type in the samples.
   Results are shown in the table below:

| | | | | | | In % of |
|---|---|---|---|---|---|---|
| | | | | In % of | | linkages |
| | | In % of | | linkages | | excluding |
| | | linkages | | excluding | In % of total | non-reducing |
| | | excluding | In % of total | non-reducing | linkages of | terminal |
| | | non-reducing | linkages of | terminal | commercialized | residues of |
| | In % of total | terminal | commercialized | residues of | Polydextrose | Polydextrose |
| | linkages of | residues of | Promitor ® 70 | Promitor ® 70 | (Litesse ® | (Litesse ® |
| | Example 1 | Example 1 | (from Tate & Lyle) | (from Tate & Lyle) | from DuPont) | from DuPont) |
| Non- | 57.0 | — | 45.0 | — | 56.4 | — |
| reducing | | | | | | |
| terminal | | | | | | |
| residue | | | | | | |
| α,β-1,2 | 6.3 | 14.7 | 4.7 | 8.6 | 3.2 | 7.3 |
| glycosidic | | | | | | |
| linkage | | | | | | |
| α,β-1,3 | 4.7 | 10.9 | 5.7 | 10.4 | 1.1 | 2.5 |
| glycosidic | | | | | | |
| linkage | | | | | | |
| α,β-1,4 | 4.7 | 10.9 | 15.3 | 27.8 | 7.0 | 16.1 |
| glycosidic | | | | | | |
| linkage | | | | | | |
| α,β-1,6 | 22.7 | 52.7 | 17.3 | 31.5 | 16.5 | 37.8 |
| glycosidic | | | | | | |
| linkage | | | | | | |
| α,β-1,3,6 | 3.7 | 8.5 | 5.0 | 9.1 | — | — |
| or 1,2,6 | | | | | | |
| glycosidic | | | | | | |
| linkage | | | | | | |
| α,β-1,4,6 | 1.0 | 2.3 | — | — | — | — |
| glycosidic | | | | | | |
| linkage | | | | | | |
| α,β-1,4,6 | — | — | 5.7 | 10.3 | — | — |
| or 1,5,6 | | | | | | |
| glycosidic | | | | | | |
| linkage | | | | | | |
| other | — | — | 1.3 | 2.4 | 15.8 | 36.2 |
| | 100.0 | 100.0 | 100.0 | 100.0 | 100.0 | 100.0 |

As can be seen in the table above, the proportion of α,β-1,6 glycosidic linkages is much higher for the glucose oligosaccharide composition according to the invention than commercially available dextrins (such as Promitor® 70 from Tate & Lyle, produced via the dextrinization of starches) and commercially available polydextrose (polymerization of glucose in the presence of acid catalyst and sorbitol) e.g. Litesse® from DuPont.

Without being bound by theory, it is thought that this increased proportion of 1,6 glycosidic linkages in the composition of the invention may be the cause of the beneficial immunological effects observed when the composition is included in an animal's diet.

### Experiment 2: In-Vitro Immune Response Trials

The immune response mechanism of the syrup Example 1 was compared to a prior art sample Example 2 in below in-vitro Experiment 2a (Nitric oxide assay to test whether compounds are capable of stimulating chicken macrophage-like cells to produce Nitric Oxide) and Experiment 2b (Phagocytosis assay to test whether compounds are able to increase phagocytosis/internalization of beads in chicken macrophage-like cells).

Example 2 is a gluco-oligosaccharide fiber obtained by an enzymatic process, as disclosed in EP0325872A1.

### Experiment 2a: Nitric Oxide (NO)-Production Assay

A nitric oxide production assay using chicken macrophage-like cell line HD11, as also described by Biggelaar et al., Vaccines 2020, 8(2), 332, was carried out to compare immune responses in terms of NO production of the glucose-oligosaccharide according to Example 1 and a prior art glucose-oligosaccharide fibre according to Example 2.

To obtain a standard NO calibration curve, the Griess assay was used as described by Biggelaar et al.

Lipopolysaccharide (LPS) were used as a positive control (leading to high NO production) and RPMI medium (i.e. Roswell Park Memorial Institute (RPMI)-1640 medium) and DMSO were used as negative controls (which both lead to zero NO production).

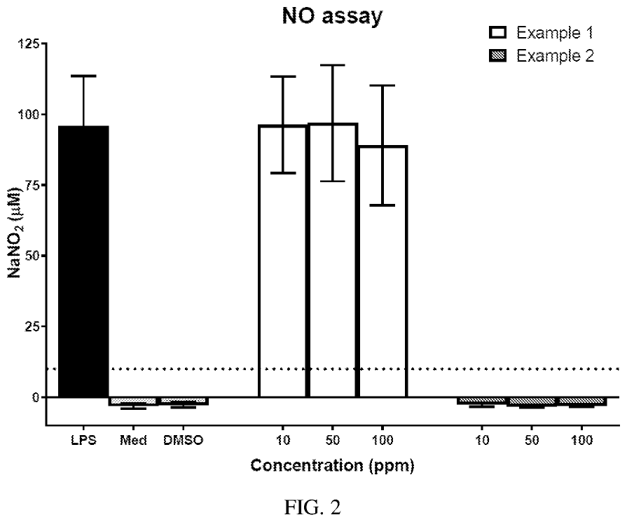

On day 1 HD11 cell were seeded, day 2 compounds were added at different concentrations i.e. at 10 ppm, 50 ppm and 100 ppm. On day 4 the supernatants were collected and the NO assay was performed giving the NO concentrations displayed FIG. 2. Compounds were tested in triplicate per NO assay i.e. in total 3 series of NO assays were performed per compound per concentration.

FIG. 2 shows that the glucose oligosaccharide according to the invention clearly has an immune response by stimulating chicken macrophage-like cells to produce nitric oxide. Macrophages produce NO as a cytotoxic and antimicrobial defense mechanism against viruses, tumor cells, bacteria, fungi, protozoa and helminths. Thus, the right amount of NO production initiates activation of the innate immune system and protects the host against infection.

(NB: Negative output implies zero NO production. Negative values arise due to the calculation based on the standard NO calibration curve. Negative output can be seen as 0 NO production.)

### Experiment 2b: Phagocytosis Assay

A phagocytosis assay using chicken macrophage-like cell line HD11, as also described by de Geus et al., Journal of Immunology 2012, 188:4516-4526, was carried out to compare immune responses of glucose-oligosaccharide according to Example 1 and a prior art glucose-oligosaccharide fibre according to Example 2 in terms of their ability to increase phagocytosis/internalization of beads in chicken macrophage-like cells.

Macrophages can internalize external bacteria and are capable of presenting these internalized bacteria to other immune cells, such as B and T cells. As a result, the innate immune system is activated, which can subsequently activate the adaptive immune system.

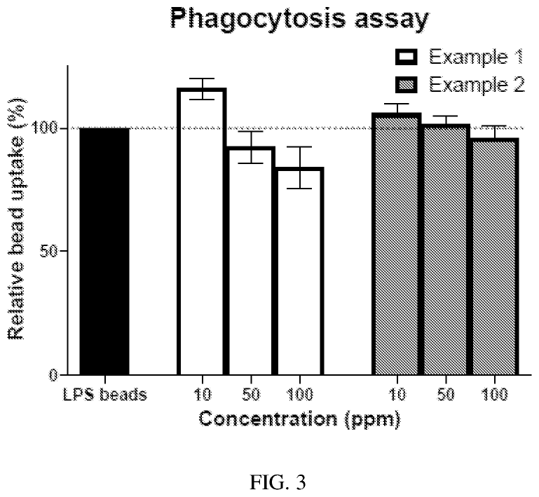

Results which show relative bead uptake at concentrations of 10, 50 and 100 ppm of the glucose-oligosaccharides (Example 1 according to the invention and Example 2 according to the prior art) are shown in FIG. 3. Clearly at 10 ppm the glucose oligosaccharide of Example 1 has a higher dose-response effect.

### Experiment 3: In Ovo Trial

In ovo trials were carried out to ensure that the glucose-oligosaccharide according to the invention at increasing concentrations does not affect mortality of unhatched chicks. The trials were thus carried out to indicate safety of the compound according to the invention in animals In this case, a comparison was made with Example 3, a commercially available conjugated linoleic acid (CLA). It is known that CLA may modulate the immune system.

480 eggs we incubated under standard conditions. They were divided unto 8 groups of 10 eggs replicated 6 times. At 17 days of incubation eggs were injected with 1 mL of the respective solution of Example 1 and Example 3 at increasing concentrations. The intention was to deliver into the amniotic fluid to be orally consumed and digested by the developing embryos according to the method described by (Uni et al., 2005, Poult Sci 84:764-770). At day of hatch, hatched chicks as well as remaining non-hatched eggs were counted and % hatchability was calculated to indicate safety of injected materials by not causing mortality.

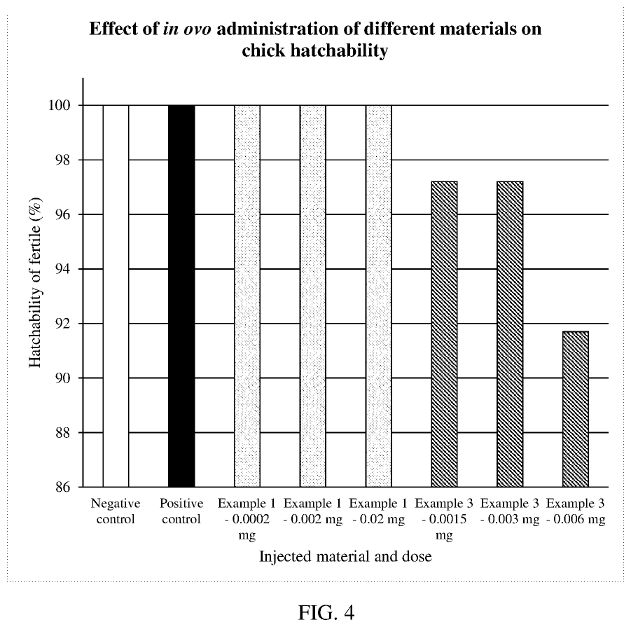

Results are shown in FIG. 4. Clearly, even at concentrations of up to 0.02mg, 100% of the chicks administered with a solution of the glucose-oligosaccharide of Example 1 hatched normally on hatching day. In comparison, a proportion of chicken eggs administered with the material of Example 3 at up to just 0.006 mg were negatively affected by the material. Not all of the chicks hatched.

This shows that Example 1 glucose-oligosaccharide according to the invention is safe to use in animals

### Experiment 4: Growth Performance Trials

### Experimental Design

Birds were fed the inventive diet at a 0.02 wt % inclusion level and a 0.2 wt % inclusion level of the glucose oligosaccharide of the invention or a control diet containing no added compounds.

Treatment diets were fed from 0 to 21 days of age. Treatments were randomly distributed per block to 144 pens, resulting in 12 replicates per treatment with 5 chickens each.

Diets

A standard starter diet was formulated. Test compound of the invention was added to the diet at the expense of ground corn (1:1 exchange in weight).

Diets were pelleted at 2.5 mm pellet length.

Composition of the experimental basal diets is given in Table 1.

TABLE 1
Ingredient composition in wt %

| | | Diet with glucose | Diet with glucose |
|---|---|---|---|
| | Reference | oligosaccharide | oligosaccharide |
| | control | (Example 1) at | (Example 1) at |
| | diet | 0.02 wt % | 0.2 wt % |
| Ground corn | 38.58 | 38.56 | 38.38 |
| Ground wheat | 20.00 | 20.00 | 20.00 |
| Soybean Meal | 36.97 | 36.97 | 36.97 |
| Salt | 0.26 | 0.26 | 0.26 |
| Calcium carbonate | 0.78 | 0.78 | 0.78 |
| Soybean oil | 0.50 | 0.50 | 0.50 |
| Choline chloride-60 | 0.08 | 0.08 | 0.08 |
| Sodium bicarbonate | 0.19 | 0.19 | 0.19 |
| DL-methionine - dry | 0.22 | 0.22 | 0.22 |
| Monocalcium | 1.41 | 1.41 | 1.41 |
| Phosphate 21% | | | |
| ENZ C | 0.010 | 0.010 | 0.010 |
| HOSTAZYM-X | | | |
| 15000 (digestibility | | | |
| enhancer endo-1,4- | | | |
| beta-xylanase) | | | |
| PHY9 AXTRA PHY | 0.004 | 0.004 | 0.004 |
| L10000 (phytase) | | | |
| Vitamin Premix | 0.150 | 0.150 | 0.150 |
| Trace Mineral Premix | 0.060 | 0.060 | 0.060 |
| Solka floc 200 Fcc | 0.797 | 0.797 | 0.797 |
| (cellulose as | | | |
| bulking agent) | | | |
| Glucose | 0 | 0.0200 | 0.2000 |
| oligosaccharide | | | |
| of Example 1 | | | |
| TOTAL | 100.0 | 100.0 | 100.0 |

### Animals, Environment, and Sample Management

Nine hundred Ross 308 male one-day-old chickens, originating from a prime 50 week aged broiler breeder flock, were purchased from a commercial hatchery and randomly allocated across 144 pens, resulting in 5 broiler chickens per pen. Broiler chickens were vaccinated for Marek and Newcastle disease at the hatchery. Pens (41×41 cm/0.17 m2) contained one tray feeder and two automatic nipple drinkers that were adjustable in height. Both feed and water were provided ad libitum throughout the study.

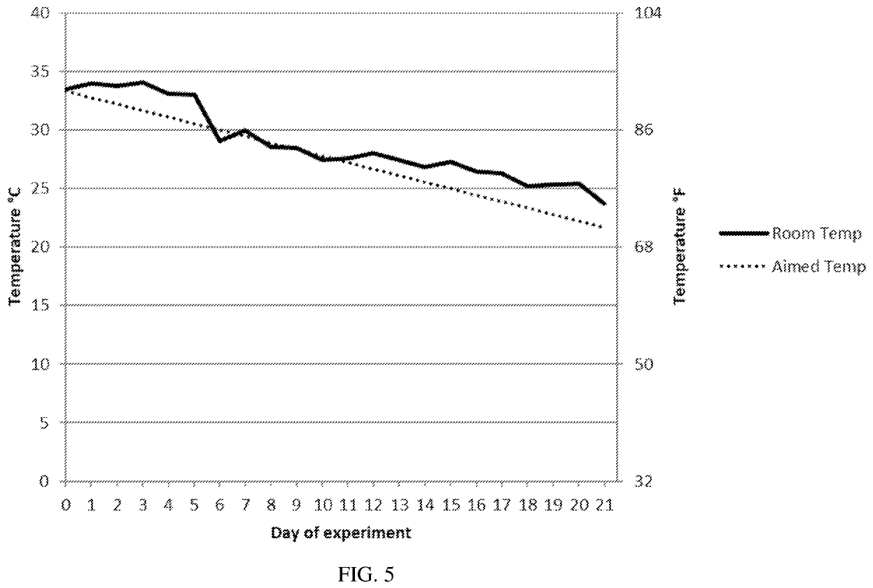

Temperature and ventilation were computer controlled. Starting at 92° F. (33.3° C.) at the day of arrival, temperature was set to gradually decrease by 1° F. (approx. 0.6° C.) per day to a final temperature of 71° F. (21.7° C.) at 21 days of age. Aimed and realized temperatures are shown in FIG. 5 (Set and measured temperatures in the Poultry Metabolic facility during the experimental period). Light was provided 23 hours per day following industry standards.

### Data Collection

Body weights per pen were recorded at 0, 7 and 21 days. In addition, feed consumption for each pen was recorded on the same day the broilers were weighed. Based on the calculated body weight gain and feed consumption, gain to feed ratio (G:F) was calculated as kg of weight gain/kg of feed consumed and corrected using the body weights of removals and mortality.

At 21 days of age, three chickens per pen (6 odd blocks) were selected and weighed individually in advance of sampling, which was done using cloaca swabs. Swabs were swirled in 0.5 ml of DNA/RNA Shield (Zymo Research, California, USA) using a 2 ml tube, releasing excreta contents from the swab into the tube. Next, tubes were stored at room temperature. Results of growth performance and mortality in broiler chickens from 0 to 21 days of age are shown in the table below.

| | | Diet with glucose | Diet with glucose |
|---|---|---|---|
| | | oligosaccharide | oligosaccharide |
| | Reference | (Example 1) at | (Example 1) at |
| | control diet | 0.02 wt % | 0.2 wt % |
| ADGc 0-21 days, g | 51.2 | 51.5 | 52.3 |
| ADFIc 0-21 days, g | 63.6 | 62.8 | 63.7 |
| G:Fc 0-21 days, g | 0.806 | 0.820 (+1.7%) | 0.822 (+2.0%) |
| Mortality | 0.00 | 0.00 | 0.00 |
| (probability | | | |
| of occurrence) | | | |

ADGc = Average daily gain corrected for within-period animal departures, arrivals and reconciliation adjustments (=(total weight out − total weight in)/number of days)
ADFIc = Average daily feed intake corrected for within-period animal departures, arrivals and reconciliation adjustments (=weight of total feed disappearance/number of days)
G:Fc = Corrected Gain to Feed ratio (=ADGc/ADFIc)

### Conclusion on Growth Performance and Mortality

It can be seen that the treatment with the glucose saccharide composition of Example 1 was given safely at two different dosages to the chickens. No increase in mortality was observed.

The “gain to feed ratio” significantly improved over the 21-day trial period. Thus, even under non-challenging conditions the diet with the compositions according to the invention improved the overall growth performance of the broiler chickens in comparison to the control diet. Best results were seen at a concentration of 0.2 wt %.

### Example 5

### In Vivo Feed Trial Immunology

A microbiota analysis in ileum/cecum was carried out by adding the glucose-oligosaccharide of Example 1 as a feed additive to the diet of Ross 308 broiler chickens (at a concentration of 0.2 wt %) from Day 0 (hatching day) until they were 21 days old.

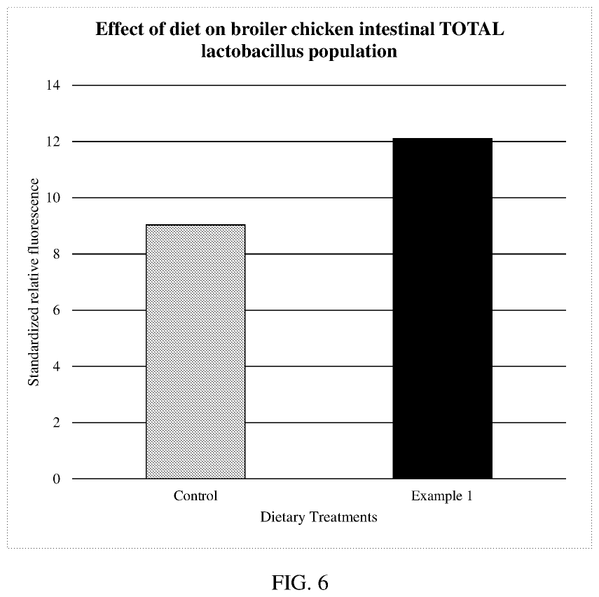

FIG. 6 shows an increase in the intestinal lactobacillus population of the chickens at age day 7 when having been fed daily with a diet including the glucose-oligosaccharide of Example 1 as a feed additive, in comparison to the control diet. In particular, significant increases in Lactobacillus 3 and Lactobacillus reuteri 1 were observed in the intestinal population, measured by fluorescent in situ hybridization (FISH) as a function of standardized relative fluorescence:

| Common_Name | Control | Example 1 |
|---|---|---|
| Lactobacillus reuteri 2 | 2.64 | 2.69 |
| Lactobacillus 3 | 3.24 | 4.77 |
| Lactobacillus reuteri 1 | 3.16 | 4.64 |

Thus, the glucose-oligosaccharide of Example 1 as a feed additive changed microbiota composition and promoted Lactobacillus species in the intestine.

1. A glucose oligosaccharide composition obtainable by a process comprising the aqueous polymerisation of glucose at a concentration of 50 to 95 wt %, preferably 70 to 90 wt %, in the presence of hydrochloric acid at a concentration of 0.01 to 0.25M, preferably 0.10 to 0.20 M, more preferably 0.10 to 0.15 M, at a temperature of from 50 to 120° C., preferably 50 to 99° C.

2. The composition according to claim 1 characterized in that it comprises of from 30 weight % (wt %) on a dry basis (db) or higher, preferably from 40 wt % db or higher, more preferably about 45 wt % db of glucose oligosaccharides having a degree of polymerisation (DP) of at least 3.

3. The composition according to claim 1 characterized in that it comprises one or more of the following:
i. glcuose in an amount of from 10 to 60 wt % db, preferably from 15 to 55 wt % db, more preferably 20 to 50 wt % db, most preferably 30 to 48 wt % db;
ii. glucose disaccharides in an amount of from 5 to 15 wt % db, preferably of from 6 to 12 wt % db, more preferably of from 7 to 10 wt % db, most preferably of from about 8 to 9 wt % db;
iii. glucose oligosaccharides having a DP of 3 in an amount of from 5 to 30 wt % db, preferably from 8 to 25 wt % db, more preferably 10 to 20 wt % db, most preferably 12 to 18 wt % db;
iv. glucose oligosaccharides having a DP of 4 in an amount of from 5 to 20 wt % db, preferably from 6 to 18 wt % db, more preferably 7 to 15 wt % db, most preferably 8 to 12 wt % db;
v. glucose oligosaccharides having a DP of 5 in an amount of from 3 to 20 wt % db, preferably from 4 to 18 wt % db, more preferably 5 to 15 wt % db, most preferably 6 to 12 wt % db;
vi. glucose oligosaccharides having a DP of 6 in an amount of from 2 to 15 wt % db, preferably from 3 to 12 wt % db, more preferably 3 to l0 wt % db, most preferably 4 to 8 wt % db;
vii. glucose oligosaccharides having a DP of 7 in an amount of from 2 to 15 wt % db, preferably from 3 to 12 wt % db, more preferably 3 to l0 wt % db, most preferably 4 to 8 wt % db;
viii. glucose oligosaccharides having a DP of 8 in an amount of from 1 to 10 wt % db, preferably from 1.5 to 8 wt % db, more preferably 2 to 6 wt % db, most preferably 2 to 4 wt % db;
ix. glucose oligosaccharides having a DP of 9 in an amount of from 0.5 to 5.0 wt % db, preferably from 0.8 to 4.0 wt % db, more preferably 1.0 to 3.0 wt % db, most preferably 1.0 to 2.0 wt % db;
x. glucose oligosaccharides having a DP greater than 9 in an amount of from 1 to 10 wt % db, preferably from 2 to 8 wt % db, more preferably 3 to 5 wt % db, most preferably 3 to 4 wt % db.

4. The composition according to claim 1 characterized in that it comprises a dry substance of at least 70 wt %, preferably at least 75 wt %, more preferably at least 80 wt %.

5. The composition according to claim 1 characterised in that at least 45%, preferably at least 50%, and at most 67%, preferably at most 65% or at most 60%, of the total number of glycosidic linkages of the glucose oligosaccharides are alpha- & beta-1,6 linkages, and/or
characterised in that at least 5%, more preferably at least 7%, and at most 15%, preferably at most 12%, more preferably at most 11% or at most 10% of the total number of glycosidic linkages of the glucose oligosaccharides are alpha- & beta-1,4 linkages, and/or
characterised in that at least 5%, more preferably at least 7%, and at most 15%, preferably at most 12%, more preferably at most 11% or at most 10% of the total number of glycosidic linkages of the glucose oligosaccharides are alpha- & beta-1,3 linkages, and/or
characterised in that at least 8%, preferably at least 10%, more preferably at least 12%, and at most 18%, preferably at most 15%, more preferably at most 14% of the total number of glycosidic linkages of the glucose oligosaccharides are alpha- & beta-1,2 linkages, and/or
characterised in that at least 4%, preferably at least 5%, and at most 12%, preferably at most 10%, of the total number of glycosidic linkages of the glucose oligosaccharides are alpha- & beta-1,3,6 or 1,2,6 linkages, wherein the “total number of glycosidic linkages” excludes linkages in the non-reducing terminal residues.

6. The composition according to claim 1 characterized in that the temperature of the polymerisation step in the process is from 60 to 98° C., preferably from 70 to 95° C., more preferably from 80 to 92° C., yet more preferably from 85 to 92° C., most preferably around 90° C.

7. The composition according to claim 1 characterized in that the reaction time of the polymerisation step in the process is from 2.5 to 40 hours, preferably 5 to 30 hours, preferably from 10 to 27 hours, more preferably from 12 to 25 hours, even more preferably from 15 to 23 hours, most preferably from 16 to 20 hours or about 18 hours.

8. An animal feed or a pet food product comprising the glucose oligosaccharide composition of claim 1 and further animal feed or pet food ingredients.

9. The animal feed or pet food product of claim 8, wherein the glucose oligosaccharide composition of is present in an amount sufficient to provide from 0.01 to 0.02 g of the composition per kg of body weight of the animal or pet per day, in one or more servings.

10. The animal feed or pet food product of claims 8 wherein the glucose oligosaccharide composition of is present in an amount of from 0.01 to 0.5 wt %, preferably from 0.01 to 0.3 wt %, more preferably from 0.02 to 0.2 wt %, based on the weight of the animal feed or pet food product.

11. (canceled)

12. (canceled)

13. A method for improving immune system modulation of animals, the method comprising feeding an animal the composition of claim 1 or an animal feed comprising the composition of claim 1.

14. A method for prophylactic treatment of animals, the method comprising feeding an animal the composition of claim 1 or an animal feed comprising the composition of claim 1.

15. The method according to claim 14 wherein the prophylactic treatment prevents, or reduces the severity of, infectious diseases, preferably diseases caused by viruses, bacteria, fungi or parasites.

16. A process for making a composition comprising glucose oligosaccharides characterized in that it comprises the steps of
a. Preparing an aqueous solution of glucose having a concentration of 50 to 95%, preferably 70 to 90% (dry substance),
b. Adding hydrochloric acid catalyst to the aqueous solution of glucose to reach a concentration of 0.01 to 0.25M hydrochloric acid, preferably 0.10 to 0.20 M, more preferably 0.10 to 0.15 M.
c. Bringing the solution to a temperature of from 50 to 120° C., preferably 50 to 99° C. to polymerise the glucose, and
d. Optionally, adjusting the pH of the glucose oligosaccharide composition to a pH of from 4 to 7.

17. The process according to claim 16 characterized in that the concentration of hydrochloric acid of the solution from step b) is from 0.02 to 1.5 M, preferably from 0.05 to 1.0 M, more preferably from 0.08 to 0.8 M, most preferably about 0.1 M.

18. The process according to claim 16 characterized in that the temperature of the polymerisation in step c) is from 60 to 98° C., preferably from 70 to 95° C., more preferably from 80 to 92° C., yet more preferably from 85 to 92° C., most preferably around 90° C.

19. The process according to claim 16 wherein the reaction time of the glucose polymerisation in step c) is from 2.5 to 40 hours, preferably 5 to 30 hours, preferably 10 to 27 hours, more preferably from 12 to 25 hours, even more preferably from 15 to 23 hours, most preferably from 16 to 20 hours or about 18 hours.
